(19) United States
(12) Patent Application Publication
(10) Pub. No.: US 2009/0192418 A1
(43) Pub. Date: Jul. 30, 2009

(54) Moving Body Inspection Apparatus And Method of Comparing Phases Between Movement Waveforms

(76) Inventors: Tsuyoshi Miyashita, Fuchu (JP); Akihiko Kandori, Tokyo (JP); Kuniomi Ogata, Tokorozawa (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/368,786

(22) Filed: Feb. 10, 2009

Related U.S. Application Data
(62) Division of application No. 11/746,144, filed on May 9, 2007, now abandoned.

(30) Foreign Application Priority Data
May 9, 2006 (JP) 2006-130124

(51) Int. Cl. A61B 5/11

(52) U.S. Cl. 600/595

(57) ABSTRACT

In a moving body inspection apparatus, sequential waveform data obtained from a movement sensor is analyzed, wherein a plurality of waveforms from the waveform data is generated; phases compared among a plurality of movement waveforms; and a result of comparing phases are displayed. A partial waveform in a frequency analysis time interval having a predetermined length is extracted from a plurality of the movement waveforms. A frequency analysis operation is performed for the extracted partial waveforms to calculate phases at maximum power frequencies in the frequency analysis time interval; a phase difference is calculated among a plurality of the movement waveforms. The peaks may be detected in a plurality of movement waveforms. Peak intervals are calculated as time intervals between adjoining peaks. The peaks among the movement waveforms are matched. Phase differences are calculated among a plurality of the movement waveforms.

### CROSS REFERENCE TO RELATED APPLICATIONS

This application is a divisional application of U.S. application Ser. No. 11/746,144, filed May 9, 2007, the contents of which are incorporated herein by reference.

This application claims the foreign priority benefit under Title 35, United States Code, §119(a)-(d) of Japanese Patent Application No. 2006-130124, filed on May 9, 2006 in the Japan Patent Office, the disclosure of which is herein incorporated by reference in its entirety.

### BACKGROUND OF THE INVENTION

1. Field of the Invention

The present invention relates to a moving body inspection apparatus and a method of comparing phases between movement waveforms and particularly to a moving body inspection apparatus for displaying quantitative movement information through analysis of waveforms obtained by a movement sensor.

2. Description of the Related Art

A method of tapping with a finger of a patient is known which quantitatively estimates decrease in a motor function due to motor paralysis. Such a method of tapping for quantitatively estimating the motor function through calculating an average of tapping intervals and a standard deviation, is disclosed by McCombe Waller S, Whitall J., “Fine Motor Control in Adults With and Without Chronic Hemiparesis: Baseline Comparison to Nondisabled Adults and Effects of Bilateral Arm Training” Arch Phys Med Rehabil 85, 1076-1083 (2004).

### SUMMARY OF THE INVENTION

An aspect of the present invention provides a moving body inspection apparatus comprising: analyzing means for analyzing time series waveform data obtained from a movement sensor, the analyzing means including: movement waveform generating means for generating a plurality of waveforms from the waveform data; and phase comparing means for comparing phases among a plurality of movement waveforms; and displaying means for displaying a result of comparing phases.

According to this structure, a detailed estimation may be provided regarding correlation among a plurality of movements because phases in a plurality of the movement waveforms can be compared.

Another aspect of the present invention provides a method of comparing phases among a plurality of the waveforms obtained from a movement sensor, comprising: the steps of: (a) extracting partial waveforms from the waveforms in a frequency analysis time interval having a predetermined time interval; (b) frequency-analyzing the partial waveforms in the frequency analysis time intervals of the waveforms and calculating phases at maximum power frequencies in the frequency analysis time intervals of the partial waveforms; (c) calculating phase differences at the maximum power frequencies in the movement waveforms; and (d) comparing phases among a plurality of the waveforms.

A further aspect of the present invention provides a method of comparing phases among a plurality of the waveforms obtained from a movement sensor, comprising: the steps of: (a) extracting peaks in a plurality of the waveforms; (b) calculating peak time differences between adjoining peaks out of the peaks; (c) matching the peaks among the movement waveforms; (d) calculating time difference among the matched peaks in the movement waveforms; (e) calculating phase differences among the movement waveforms on the basis of the time differences among the matched peaks; and (f) comparing the phases among a plurality of the waveforms.

### BRIEF DESCRIPTION OF THE DRAWINGS

The object and features of the present invention will become more readily apparent from the following detailed description taken in conjunction with the accompanying drawings in which:

FIG. 1 is a block diagram of a moving body inspection apparatus according to first and second embodiments of the present invention;

FIG. 2A is a block diagram of an example of a movement sensor according to first and second embodiments of the present invention;

FIG. 2B is an illustration showing both hands provided with transmitting coils and receiving coils;

FIG. 3 is a block diagram of an analysis processing section according to a first embodiment;

FIGS. 4A and 4B are charts for explaining a process of extracting waveform data in a plurality of frequency analysis time intervals from distance waveforms;

FIG. 5A is a chart for showing a movement waveform of a channel one;

FIG. 5B is a chart for showing a movement waveform of a channel two;

FIG. 5C is a chart for showing a phase difference waveform between the movement waveform of the channels one and two;

FIG. 6 is a chart of power spectrum Aⁿ (s,f) for each short time interval;

FIGS. 7A, 7B, and 7C are charts for showing correlation of feature quantities of movements;

FIG. 8 is a flowchart of a phase comparing process in the moving body apparatus according to the first embodiment;

FIG. 9 is a block diagram of an analysis processing section according to a second embodiment;

FIGS. 10A, 10B, and 10C are charts for explaining a process of calculating peak intervals by a peak interval calculating part;

FIGS. 11A and 11B are charts for explaining a process of matching peaks between two movement waveforms;

FIG. 12 is a flowchart of a phase comparing process in the moving body inspection apparatus according to the second embodiment;

FIG. 13 is an illustration of an example of a screen image displayed on a display by a display processing part according to the first and second embodiment;

FIGS. 14A and 14B are charts for showing examples of analysis results displayed in a type one analysis result display area according to the first embodiment;

FIGS. 15A and 15B are charts for showing examples of analysis results displayed in a type two analysis result display area according to the second embodiment; and

FIGS. 16A and 16B are enlarged charts for showing parts in FIGS. 15A and 15B.

The same or corresponding elements or parts are designated with like references throughout the drawings.

### DETAILED DESCRIPTION OF THE INVENTION

Prior to describing an embodiment of the present invention, the above-mentioned related art will be further explained.

The method disclosed in the above-mentioned prior art document (McCombe Waller S, Whitall J.) cannot analyze difference in phase of tapping timing between both hands in time-series manner. Thus it is difficult to provide correlation of movements of both hands in detail.

The present invention intends to provide a moving body inspection apparatus and a method of comparing phase between movement waveforms in phase.

With reference to drawings will be described preferred embodiments of the present invention.

### First Embodiment

In a first embodiment, phases are compared among a plurality of movement waveforms through frequency-analyzing for each of a plurality of movement waveforms.

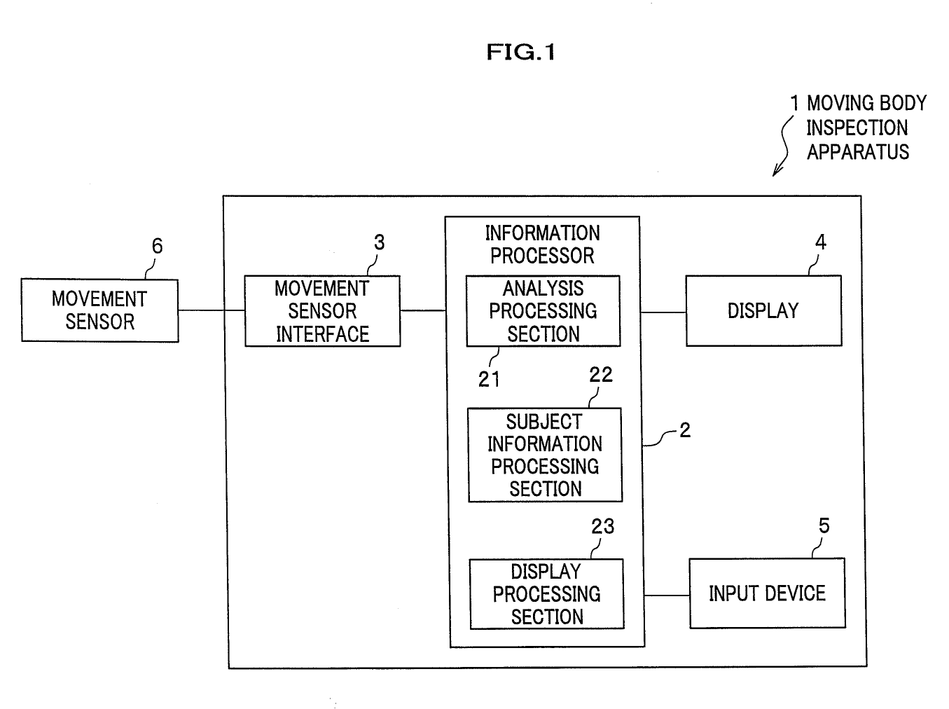

FIG. 1 is a block diagram of a moving body inspection apparatus 1 according to the first embodiment.

As shown in FIG. 1, the moving body inspection apparatus 1 includes information processor 2, a movement sensor interface 3, a display 4, and an input device 5.

A movement sensor 6 for obtaining movement information of a subject as waveform data is connected to the moving body inspection apparatus 1 through the movement sensor interface 3 in the moving body inspection apparatus 1. The movement sensor 6 is a sensor for detecting movement information of the subject and thus any movement sensor can be used as long as it can obtain, as waveform data, movement information of the subject corresponding to at least one of a distance, a velocity, an acceleration, and a jerk.

The “subject” is a target to be measured with the movement sensor 6 and may be anything moving such as a machine, an animal, a human being, and the like. Unless otherwise specified, the embodiments of the present invention exemplifies a case where a subject has a disorder in the motor function due to, for example, cerebral infarction, Parkinson's disease patients, and cervical spine losis patients.

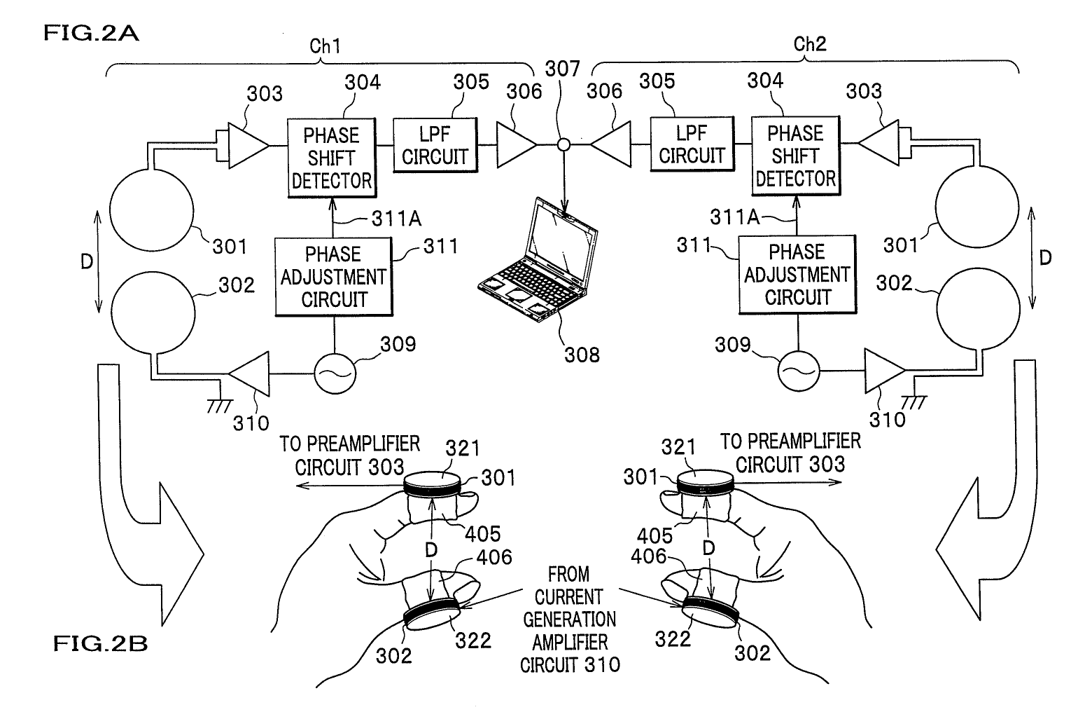

FIG. 2A is a block diagram of an example of the movement sensor 6 according to the embodiments of the present invention. As shown in FIG. 2A, the movement sensor 6 is, for example, a tapping device of a magnetic sensor type. The tapping device supplies waveform data obtained by tapping units (channel one and channel two) having the same structure attached to both hands of the subject to send waveform data of the channels one and two to a computer 308. Thus, in the first and second embodiments of the present invention, the tapping unit of the channel one is mainly described as the movement sensor 6, and a duplicated description will be omitted.

In FIG. 2B, a transmitting coil 302 is attached to a dorsal surface of the thumb, and a receiving coil 301 is attached to a dorsal surface of the index finger. As shown in FIG. 2B, the transmitting coil 302 is formed by winding a wire around a coil bobbin 322, the wire being connected to a current generating amplifier circuit 310. The receiving coil 301 is formed by winding a wire around a coil bobbin 321, the wire being connected to a preamplifier circuit 303.

The coil bobbins 321 and 322 for receiving coil 301 and the transmitting coil 302 are attached to the fingers with bands 405 and 406, respectively, in which the bands 405 and 406 are made of elastic member such as a rubber or sponge rubber.

An AC voltage generating circuit 309 generates an AC voltage having a predetermined frequency (for example, 20 kHz). The current generating amplifier circuit 310 converts the AC voltage into an alternating current having the predetermined frequency which is supplied to the transmitting coil 302. The transmitting coil 302 generates a magnetic field according to the alternating current. The generated magnetic field generates an induced voltage in the receiving coil 301.

The induced voltage, having the same frequency as the AC voltage generated by the AC voltage generating circuit 309 is amplified by the preamplifier 303. The amplified signal is applied to a phase shift detector 304.

For detection with the AC voltage, having the predetermined frequency or a double frequency generated by the AC voltage generating circuit 309, the AC voltage of the AC voltage generating circuit 309 is phase-adjusted by a phase adjusting circuit 311, and then, applied to a reference signal input terminal of the phase detector 304 as a reference signal 311A.

In a case that the phase detection is performed with double the predetermined frequency, the phase adjusting circuit 311 is not always necessary. As a simple circuit structure for phase detection with the double frequency, a circuit is usable in which the AC voltage generating circuit 309 is set to the double frequency, which is frequency-divided by two with a frequency divider (not shown). The frequency-divided signal is applied to the current generating amplifier 310. On the other hand, the double frequency is applied to the reference signal input terminal of the phase detector 304.

The output of the phase detector 304 is low-pass-filtered with a low pass filter (LPF) circuit 305 and amplified with an amplifier 306 to have a desired voltage level to generate an output 307. The output 307 represents a voltage corresponding to a relative distance D between the receiving coil 301 and the transmitting coil 302 attached to the subject.

The output 307 is converted into digital data with an analog-to-digital conversion board built in the computer 308 and entered into the computer 308.

In the first and second embodiments of the present invention, the subject has a task of performing a tapping operation, for example, tapping the index finger on the thumb in both hands for 20 seconds as quickly as the subject can (in-phase movement).

Further, the patient is subject to a task of performing a tapping operation, for example, tapping the index finger on the thumb alternately between both hands for 20 seconds as quickly as the patient can (anti-phase movement).

The movement sensor 6 according to the first and second embodiments obtains the movement information as waveform data which can be converted into a distance waveform. In the first and second embodiments, two pieces of the waveform data measured by the tapping devices of the channel one and the channel two are obtained in any task. Thus, the embodiment is described with assumption that a plurality of pieces of waveform data are obtained by measurement with the tapping devices of the channel one and the channel two in parallel at the same time zone. However, this invention is not limited to such an embodiment that a plurality of pieces of waveform data are measured at the same time.

The information processor 2 shown in FIG. 1 analyzes waveform data obtained by the movement sensor 6 to extract a feature quantity and occasionally displays the extracted feature quantity in combination with subject information on the display 4.

The movement sensor interface 3 includes, for example, an analog-to-digital conversion board which may be installed in a general computer to convert the waveform data of an analog signal detected by the movement sensor 6 into a waveform of a digital signal with a predetermined sampling frequency S_{f} to apply the waveform data of the digital signal to the information processor 2.

Further, the sampling frequency S_{f} is also used to extract the waveform in the frequency analysis time intervals from the movement waveform (mentioned later).

The display 4 displays the subject information and the movement information processed by the information processor 2. For example, an LCD (Liquid Crystal Display) is usable as the display 4.

The input device 5 is provided for an operator to enter the subject information and instruct the information processor 2 to conduct measurement and analysis and the like. For example, a keyboard and a mouse are usable as the input device 5. In addition, in a case where the operator enters the subject information or the like or instructs the information processor 2 to conduct the measurement and the analysis, it is possible to display an input screen image on the display 4.

The information processor 2 includes an analysis processing section 21, a subject information processing section 22, and a display processing section 23. The information processor 2 is provided with a CPU (Central Processing Unit) and a memory including a ROM (Read Only Memory), a RAM (Random Access Memory) and a hard disk drive and the like. The analysis processing section 21, the subject information processing section 22, and the display processing section 23 are operated by that the CPU reads programs and data stored in the memory and the hard disk drive and load the data on the memory to execute the process.

### <Analysis Processing Section>

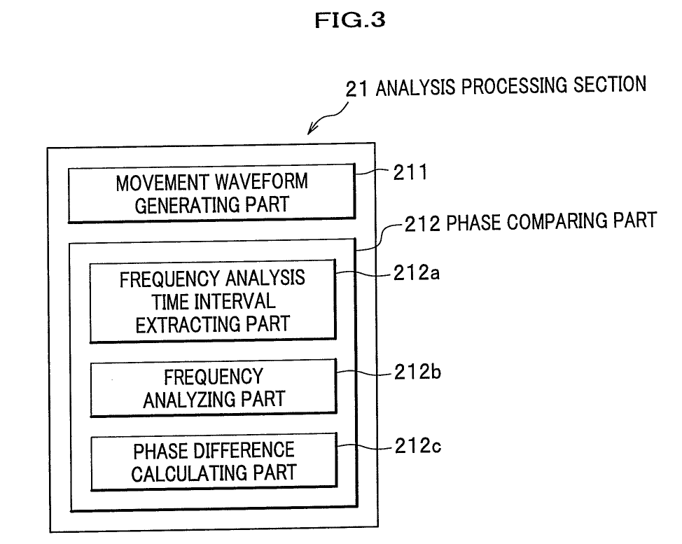

FIG. 3 is a block diagram of the analysis processing section 21 according to the first embodiment. The analysis processing section 21 extracts the feature quantity of the movement on the basis of the waveform data supplied from the movement sensor 6. The result of analysis by the analysis processing section 21 is recorded on a subject data database (not shown) installed in the subject information processing section 22 and is occasionally read from the subject data database by the display processing section 23 to be displayed on the display 4.

As shown in FIG. 3, the analysis processing section 21 includes a movement waveform generating part 211 and a phase comparing part 212.

### <Movement Waveform Generating Part>

The waveform data obtained from the movement sensor 6 is not data directly representing the movement waveform, but an output voltage convertible into a movement waveform.

The movement waveform generating part 211 converts the waveform data as the output voltage into a corresponding movement waveform and performs time differentiation or time integration to complementarily generate a distance waveform, a speed waveform, an acceleration waveform, and a jerk waveform.

The “movement waveform” includes at least one of the distance waveform, the speed waveform, the acceleration waveform, the jerk waveform, and waveforms that can be converted into the four types of the above-mentioned movement waveforms (the distance waveform, the speed waveform, the acceleration waveform, and the jerk waveform). More specifically, the movement waveform to be analyzed by the moving body inspection apparatus 1 is a waveform that can be obtained on the basis of the waveform data measured by the movement sensor 6. For example, the waveform may include those measured by the movement sensor 6 or at least one of the four types of the movement waveforms converted or complimentarily generated from the waveform data (the distance waveform, the speed waveform, the acceleration waveform, and the jerk waveform).

A time interval T of the movement waveform is a measurement time of the movement sensor 6. For example, in a case where the tapping movement of the subject is measured for 20 seconds, the time interval T is 20 seconds.

### <Phase Comparing Part>

The phase comparing part 212 compares phases among a plurality of movement waveforms obtained on the basis of a plurality of pieces of the waveform data.

In the first embodiment, the phase comparing part 212 conducts a frequency analysis operation for each of a plurality of movement waveforms to calculate and detect a phase of a maximum power spectrum (hereinafter referred to as maximum power frequency) and compares phases among a plurality of the movement waveforms by comparing the maximum power frequencies.

The phase comparing part 212 according to the first embodiment includes a frequency analysis time interval extracting part 212a, a frequency analyzing part 212b, and a phase difference calculating part 212c.

### <Frequency Analysis Time Interval Extracting Part>

The frequency analysis time interval extracting part 212a extracts a partial waveform in the movement waveform frequency analysis time interval having a predetermined time interval T₀ be frequency-analyzed by the frequency analyzing part 212b.

In this operation, the longer the time interval T₀ of the frequency analysis time interval for extraction is extended, the higher the accuracy in the frequency analysis in each frequency analysis time interval becomes. On the other hand, the shorter the time interval T₀ of the frequency analysis time interval for extraction, the more finely the information such as the phases of the maximum power frequencies as results of the frequency analysis can be calculated on time base. Thus, it is preferable to select an appropriate length of the time interval T₀ of the frequency analysis interval to be extracted. The first embodiment is explained in which the frequency analysis time interval is assumed to be 10 seconds for the movement waveform having duration of 20 seconds.

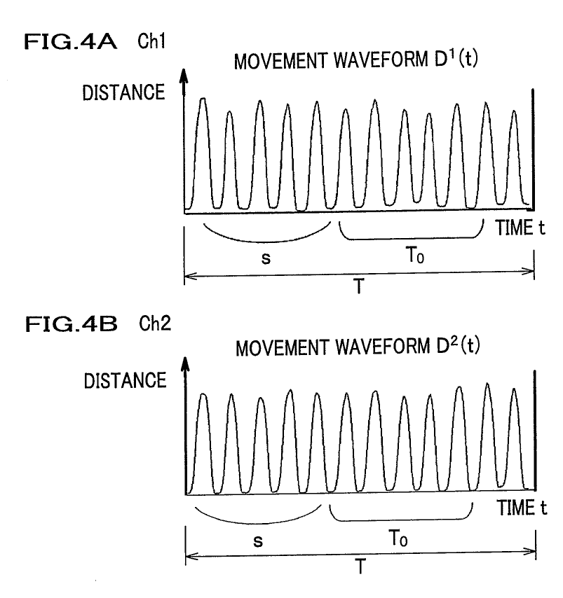

With reference to FIG. 4, will be described a process of extracting a partial waveform in frequency analysis time interval from the distance waveform as one example of the movement waveforms by the frequency analysis time interval extracting part 212. FIGS. 4A and 4B are charts for explaining the process of extracting the partial waveforms in a plurality of frequency analysis time intervals of the distance waveform to show the distance waveforms obtained in the channel one and the channel two, respectively.

Here, the analysis of the distance waveform is similarly applicable to analyses of the other movement waveforms, and thus, instead of “distance waveform” the term “movement waveform” is used as a dominant conception. Further, the processes of extracting the partial waveforms in the frequency analysis time intervals from the waveform D¹(t) of the channel one and the waveform D²(t) of the channel two are the same, and thus, the explanation is made for the movement waveform Dⁿ(t) without any distinction between the waveform D¹(t) of the channel one and the waveform D²(t) of the channel two.

First, the frequency analysis time interval extracting part 212a has a discrete expression of the movement waveform Dⁿ(t). The movement waveform Dⁿ(t) discretely expressed can be represented by the following Equation (1).

Dⁿ(t)=Dⁿᵢ (1)

where “n” is channel number,

i=1, . . . , L_{T}, and

(the number L_{T} of time intervals of movement waveform)=(the time interval T of the movement waveform)×(sampling frequency S_{f}).

In FIGS. 4A and 4B, the movement waveforms Dⁿ (t) are shown. However, to actually extract the partial waveform in the frequency analyzing time interval, a digitalized movement waveform Dⁿ is used.

Next, the frequency analysis interval extracting part 212a extracts the partial waveform in the frequency analysis time interval Dⁿ_{u,i} of the predetermined time interval T₀ from digitalized waveform Dⁿ. The partial waveform data Dⁿ_{u,i} extracted in the frequency analysis time interval is represented by equation (2).

Dⁿ_{u,i}=Dⁿᵢ (2)

where i=1, . . . , L_{T0},

j=u+i

(the number u of time intervals up to the frequency analysis time interval)=(the time interval “s” up to the frequency analysis time interval)×(sampling frequency S_{f}), and

(the number L_{T0} of time intervals in frequency analysis time interval)=(the time interval T₀ of the movement waveform)×(sampling frequency S₀).

In other words, in Equation (2), the partial waveform in the frequency analysis time interval Dⁿ_{u,i} having the predetermined time interval T₀ is successively extracted while the frequency analysis time interval Dⁿ_{u,i} is shifted by a time interval (short interval) of 1/(sampling frequency S_{f}). The partial waveforms Dⁿ_{u,i} in the frequency analysis time intervals extracted by the frequency analysis time interval extracting part 212a are applied to the frequency analyzing part 212b.

### <Frequency Analyzing Part>

The frequency analyzing part 212b performs a frequency analysis of the extracted partial waveforms Dⁿ_{u,i} in each frequency analysis time interval and calculates phases of the maximum power frequency in each frequency analysis time interval.

Next, will be described a process of frequency analysis of the partial waveform in the frequency analysis time interval Dⁿ_{u,i} by the frequency analyzing part 212b.

First, the frequency analyzing part 212b calculates power spectrum Aⁿ_{u,k} and a phase Θⁿ_{u,k} in each frequency analysis time interval Dⁿ_{u,i}, for example, by a digital Fourier Transform. The process by the digital Fourier Transform is given by Equation (3).

Waveform Dⁿ_{u,i} in Frequency Analysis Time Interval→Power Spectrum Aⁿ_{u,k} Phase Θⁿ_{u,k} (3)

where k=1, . . . , L_{f},

(the number L_{f} of digitizing in frequency base)=(L_{T0}/2), and (k/T₀=frequency).

Next, the frequency analyzing part 212b obtains the power spectrum Aⁿ_{u,k} and phase Θⁿ_{u,k} for each “u” satisfying [0≦u≦L_{T}−L_{T0}].

The frequency analyzing part 212b searches a frequency k of a maximum of the power spectrum Aⁿ_{u,k} at each time “u” and sets k(u) to the searched frequency.

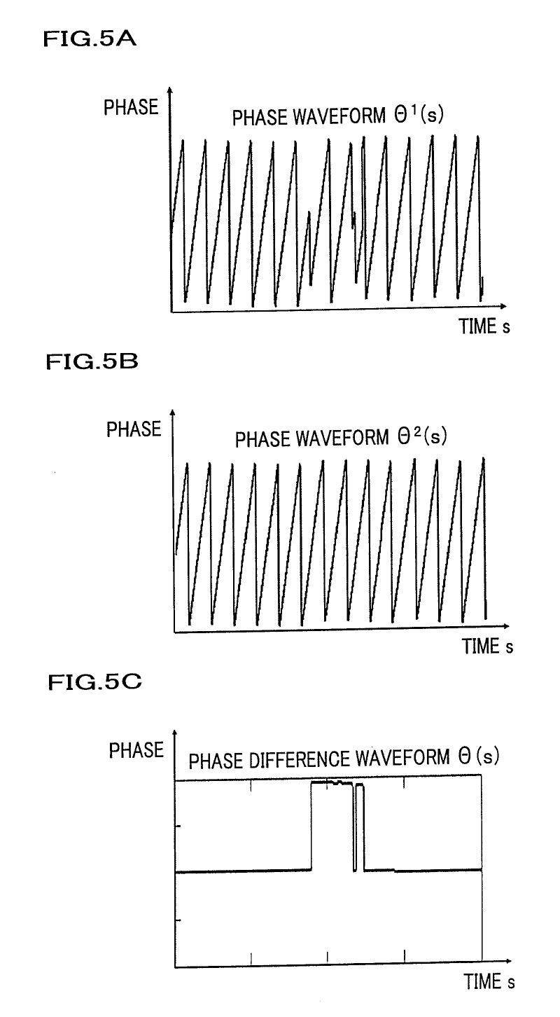

Next, the frequency analyzing part 212b determines the phase Θⁿ_{u,k(u)} at the frequency k(u) as the phase Θⁿ_{u,k} of the maximum power frequency at each of time u. At the phase Θⁿ_{u,k} of the maximum power frequency, because “u” is derived by digitizing the time interval (start timing of the frequency analysis time interval) “s” up to the frequency analysis time interval as shown in Equation (2), Θⁿᵤ=Θⁿ(s), which is represented as the phase waveforms Θⁿ (s) as shown in FIGS. 5A and 5B. FIG. 5A shows a phase waveform Θⁿ¹(s) of the channel one, and FIG. 5B shows a phase waveform Θⁿ¹(s) of the channel two.

The phase Θⁿ(s) of the maximum power frequency calculated by the frequency analyzing part 212b is applied to the phase difference calculating part 212c.

### <Phase Difference Calculating Part>

The phase difference calculating part 212c compares the phases of the maximum power frequency obtained for a plurality of the movement waveforms Dⁿ(t) and calculates a phase difference Θ(s) of the maximum power frequency among a plurality of the movement waveforms.

For example, in a case that phases are compared between two movement waveforms using the movement sensor 6 including tapping devices of the channels one and two, the phase difference Θ(s) at the maximum power frequencies can be obtained from Equation (4).

Phase difference at the maximum power frequencies Θ(s)=(Phase Θ²(s) at Maximum power frequency of Ch2)−(Phase Θ¹(s) at Maximum power frequency of Ch1) (4)

The phase difference Θ(s) at the maximum power frequency is shown as a phase difference waveform Θ(s) as shown in FIG. 5C.

Further, in a phase difference Θ(s) at the maximum power frequencies in more than two movement waveforms, for example, one movement waveform (for example, the phase at the maximum power frequency in the waveform data D¹(t) is determined as a reference, and differences between the reference and the phase at of the maximum power frequencies in the waveform data D²(t) and D³(t) are calculated, respectively.

In addition to the phase Θⁿ(s) of the maximum power frequency, the frequency analysis such as a general digital Fourier Transform conducted by the frequency analyzing part 212b can calculate the maximum power frequency and a power at the maximum power frequency.

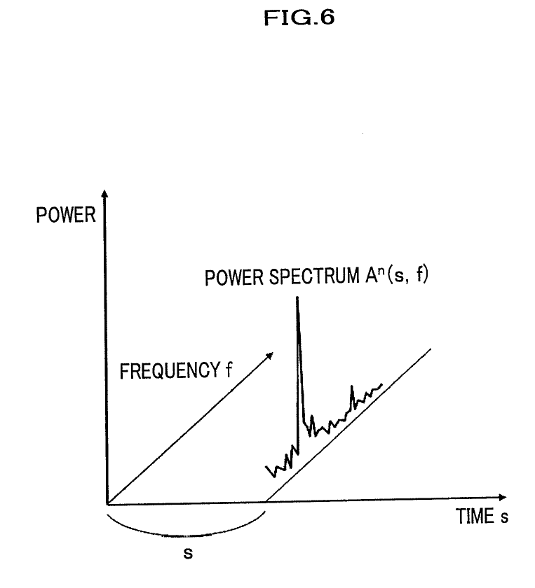

In other words, the frequency analysis with the moving body inspection apparatus 1 according to the first embodiment provides a power spectrum Aⁿ (s,f) in each short time interval for each of the movement waveforms as shown in FIG. 6. The frequency analyzing part 212b can calculate various feature quantities of movements in addition to the phase at the maximum power frequency with the power spectrum Aⁿ(s,f) for each short time interval. For example, the frequency analyzing part 212b can obtain feature quantities such as the time corresponding to the frequency analysis time interval (for example, a time interval “s” up to the frequency analysis interval), and the frequency “f” and the spectrum power from the power spectrum Aⁿ(s,f) for each short time interval. Further, the frequency analyzing part 212b can calculate feature quantities such as the maximum power frequency, the power at the maximum power frequency, and the time interval corresponding to the frequency analysis time interval from the power spectrum Aⁿ(s,f).

### <Subject Information Processing Section>

Returning to FIG. 1, the subject information processing section 22 has the subject data database (not shown) for recording the subject information and information of analysis results to manage the information recorded in the subject data database.

More specifically, the subject information processing section 22 performs mainly four processes, in combination with the subject data database, including: 1) registration, correction, deletion, searching, and sorting of the subject information; 2) relating the subject information to the movement waveform; 3) registration, correction, and deletion of analysis result of the movement waveform (including addition, correction, and deletion of items); 4) registration, correction, and deletion of results of the statistical processing in a case of conducting statistical processing.

Among subject information registered in the subject data database are a subject ID, a name, a birth date, an age, a body height, a weight, a disease name, a comment regarding the subject and the like.

This information management by the subject information processing section can be easily provided by using well-known programs and data formats.

Further, the subject data database is provided by using a hard disk drive and the like.

### <Display Processing Section>

The display processing section 23 displays information such as the subject information and the analysis results of the movement waveforms registered in the subject data database on the display 4 in a display format which is easy to be visually understandable by occasionally using charts and tables. In addition to the generation and displaying the phase waveform Θⁿ(s) and the phase difference waveform Θ(s) shown in FIGS. 5A to 5C, the display processing section 23 can generate and display, for example, a correlation chart including at least two of the time interval corresponding to the frequency analysis time interval (for example, the time interval “s” up to the frequency analysis time interval); the frequency “f”; and the spectrum intensity, and can generate and display a correlation chart including at least two of the maximum power frequency, the intensity at the maximum power frequency, and the time interval “s” corresponding to the frequency analysis time interval.

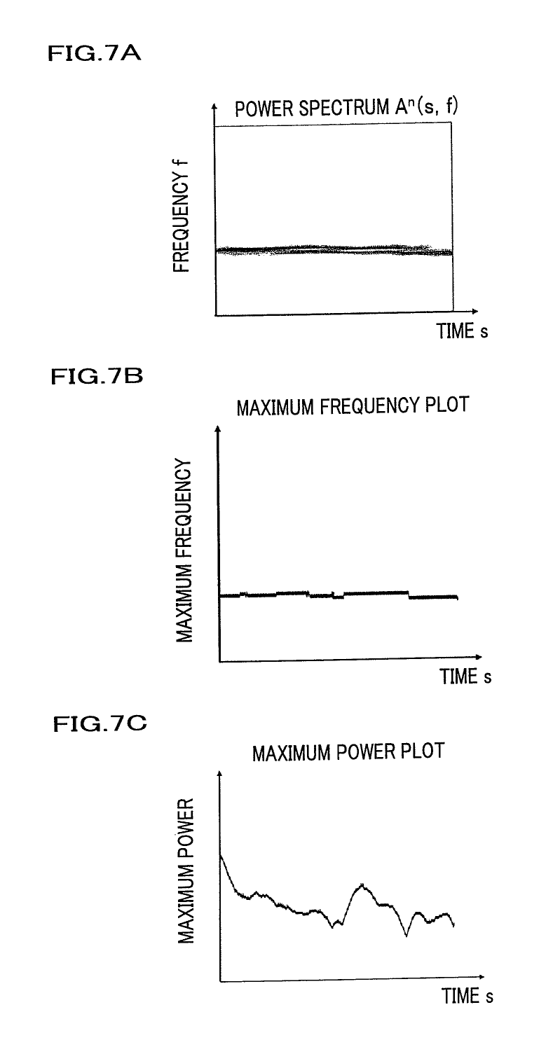

FIG. 7A is a chart showing a correlation among three feature quantities including the time interval “s” up to the frequency analysis time interval, the frequency “f”, and the power spectrum. The chart shown in FIG. 7A is actually displayed on the display 4 by not black and white but a brightness in each color in which variation in the brightness represents intensity at the frequency. FIG. 7B is a chart showing a correlation between two features of the time interval “s” up to the frequency analysis time interval and the maximum power frequency. FIG. 7C is a chart showing a correlation between two feature quantities including the time interval “s” up to the frequency analysis time interval and the power at the maximum power frequency (“Maximum Power Plot” is shown in FIG. 7C).

### [Method of Comparing Phases]

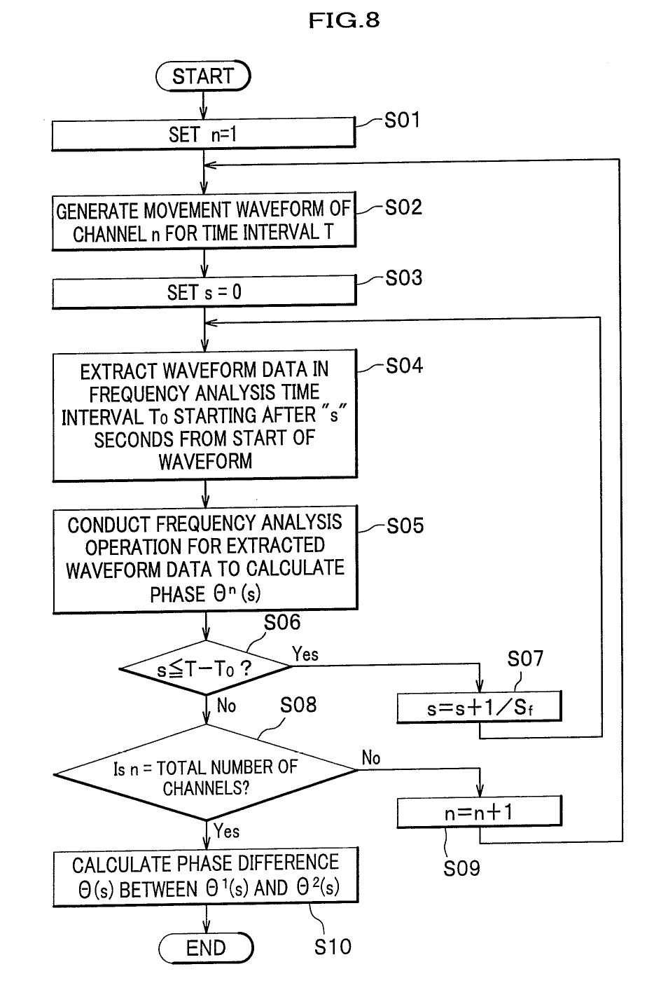

With reference to FIGS. 3 and 8, will be described an example of a method of comparing phases among a plurality of movement waveforms in the moving body inspection apparatus 1 according to the first embodiment. FIG. 8 is a flowchart of the phase comparing process in the moving body inspection apparatus 1 according to the first embodiment.

First, the movement waveform generating part 211 in the analyzing processing section 21 sets N=1, before analyzing the waveform data obtained by the “n” channel of the movement sensor 6 (see FIG. 1) (step S01).

Next, the movement waveform generating part 211 in the analyzing processing section 21 generates a movement waveform for time interval T on the basis of the “n” channel of waveform data (step S02). As mentioned earlier, the time interval T is a measurement time interval for the movement sensor 6.

The movement waveform generating part 211 in the analyzing processing section 21 sets s=0, before the frequency analysis time interval extracting part 212a extracts the partial waveform in the frequency analysis time interval after “s” seconds from the start (step S03).

Next, the phase comparing part 212 in the analyzing processing section 21 extracts the partial waveform in the frequency analysis time interval starting after “s” seconds after the start of the waveform for time interval T₀ with the frequency analysis time interval extracting part 212a (step S04).

Next, the phase comparing part 212 in the analyzing processing section 21 conducts a frequency analysis operation for the partial waveform in the frequency analysis time interval with the frequency analyzing part 212b to calculate the phase Θⁿ (s) of the maximum power frequency (step S05). For example, the frequency analysis operation is the digital Fourier Transform.

Next, the phase comparing part 212 in the analyzing processing section 21 determines whether s≦T−T₀ can be established with the frequency analyzing part 212b (step S06). If Yes in the step S06, s=s+1/Sf is calculated (step S07) and processing results to the step S04. On the other hand, if s>T−T₀ (No, in the step S06), processing proceeds to a step S08.

The phase comparing part 212 in the analyzing processing section 21 determines whether n=total number of channels (step S08). If “n” is not equal to the total number of channels, the phase comparing part 212 sets (n=n+1) (step S09) and returns to the step S02 to repeat the process from the step S02 to the step S09. On the other hand, if “n” is not equal to the total number of channels, the phase comparing part 212 proceeds to a step S10.

The phase comparing part 212 in the analyzing processing section 21 calculates the phase difference Θ(s) at maximum power frequencies between channels with the phase calculating part 212c (step S10). For example, as described above, if the total number of channels is two, the phase difference Θ(s) can be calculated in accordance with Equation (4).

### Second Embodiment

With reference to drawings will be described a second embodiment. In the second embodiment, peaks are extracted for each of the movement waveforms, and phases of the peaks are compared among a plurality of movement waveforms on the basis of the time difference of the peaks.

The second embodiment is different from the first embodiment in the structure of the analysis processing section 21. Thus, in the second embodiment will be mainly described a phase comparing part 312 in the analysis processing section 21, and thus a duplicated description will be omitted.

### <Analysis Processing Part>

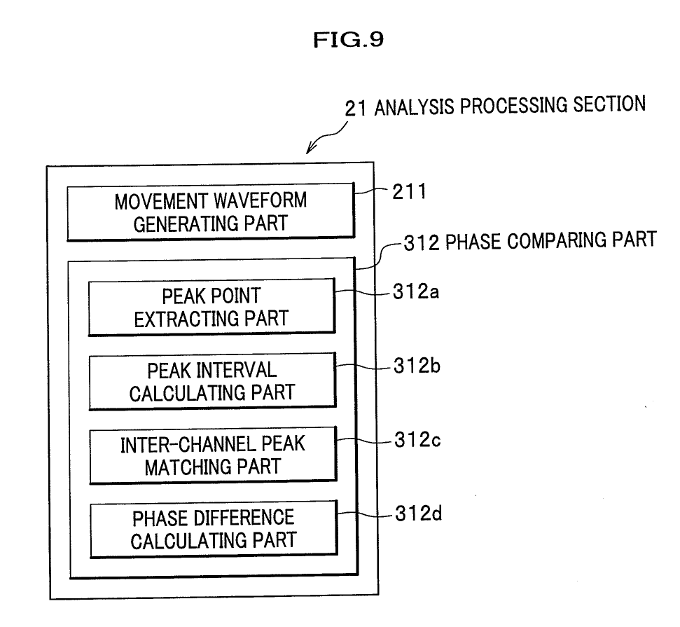

FIG. 9 is a block diagram of the analysis processing section 21 according to the second embodiment. The analysis processing section 21 includes the movement waveform generating part 211 and the phase comparing part 312.

The phase comparing part 312 according to the second embodiment includes a peak point extracting part 312a, a peak interval calculating part 312b, an inter-channel peak matching part (corresponding to an inter-movement waveform peak matching part) 312c, and a phase difference calculating part 312d.

### <Peak Point Extracting Part>

The peak point extracting part 312a extracts peak points (peaks) (1, . . . , Mⁿ; Mⁿ corresponding to the number of the peak points) in the movement waveform.

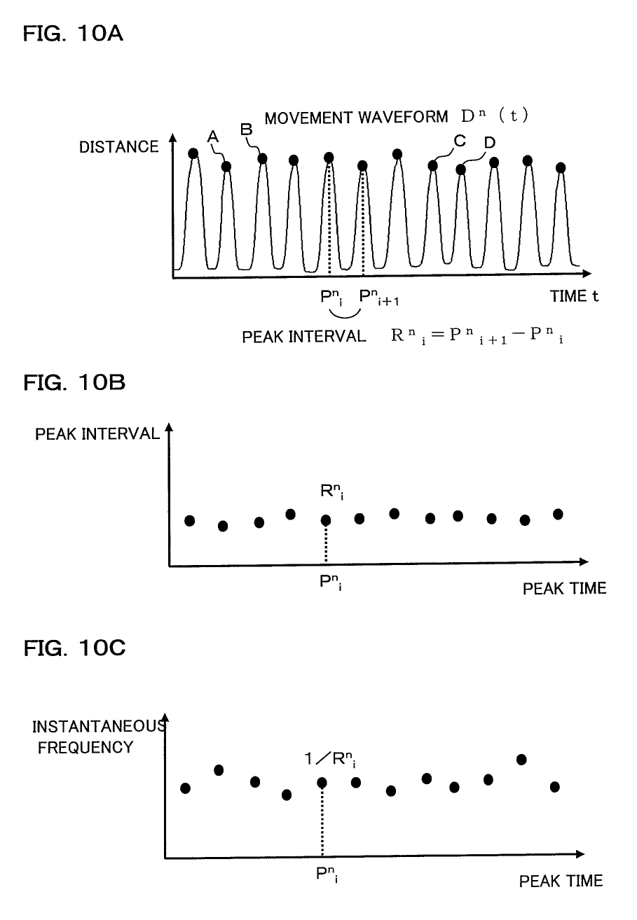

In FIG. 10A, the peak points having movement values (distance values) equal to or greater than a predetermined value are represented by black circles (). However, the peak point extracting part 312a may be configured to extract peak points having values equal to or smaller than a predetermined value. In addition, the peak point extracting part 312a may be configured to extract peak points having both values equal to smaller than and equal to or greater than the predetermined value.

The peak points (1, . . . , Mₙ) extracted by the peak point extracting part 312a are applied to the peak interval calculating part 312b and the inter-channel peak matching part 312c.

### <Peak Time Difference Calculating Part>

The peak interval calculating part 312b calculates a peak time difference which is a difference in time between adjoining peak points in one movement waveform. In FIG. 10A, as the adjoining peaks in time, there are peak points A and B, and C and D. A peak time difference Rⁿᵢ can be calculated with Equation (5).

Rⁿᵢ=Pⁿᵢ₊₁−Pⁿᵢ (i=1, - - - , (Mⁿ−1)) (5)

FIG. 10B is a plotted chart showing a correlation between the peak time difference Rⁿᵢ and the peak time of peak point which is one of two variations used for calculating the peak time differences Rⁿᵢ (for example, Pⁿᵢ).

FIG. 10C is a plotted chart showing a correlation between (1/peak time difference Rⁿᵢ) and the peak time of peak points which is one of sets of peaks used for calculating the peak time differences Rⁿᵢ (for example, Pⁿᵢ). Here, (1/peak time difference Rⁿᵢ) corresponds to an instantaneous frequency at the peak timing (hereinafter referred to as “instantaneous frequency”).

Next, the peak time difference Rⁿᵢ calculated by the peak time difference calculating part 312b is applied to the phase difference calculating part 312d.

### <Inter-Channel Peak Matching Part>

The inter-channel peak matching part 312c is provided for matching peak points among a plurality of movement waveforms.

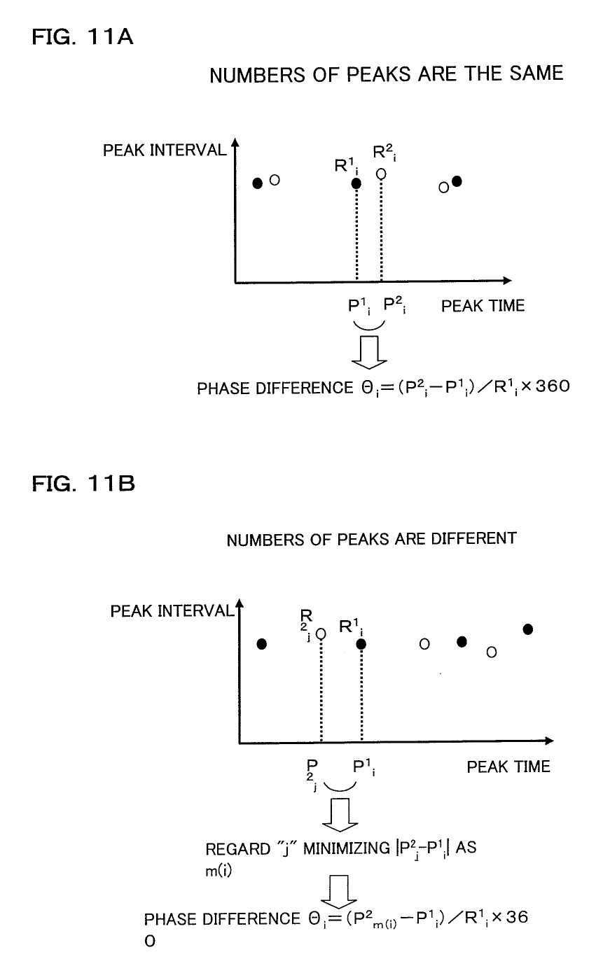

With reference to FIGS. 11A and 11B, will be described a process performed by the inter-channel peak matching part 312c to match peaks between two movement waveforms obtained from the channels one and two. FIGS. 11A and 11B are charts for explaining the process of matching the peak points between two movement waveforms.

FIG. 11A shows a case where two movement waveforms have the same number Mⁿ of peaks, and FIG. 11B shows a case where two movement waveforms have the different number Mⁿ of peaks, In FIGS. 11A and 11B, black circles () represent peak points in the movement waveform of the channel one, and circles (◯) represent peak points in the movement waveform of the channel two.

As shown in FIG. 11A, in the case where the number Mⁿ of peaks extracted from two movement waveforms (in FIG. 11A, the number of peaks are three, respectively), the inter-channel peak matching part 312c sequentially matches the peak points between the movement waveforms.

More specifically, in FIG. 11A, the inter-channel peak matching part 312c matches the peak point P²ₘ₍ᵢ₎ to the peak point P¹ᵢ by setting (m (i)=i (i=1, . . . , M¹)). Then, information of the peak points matched between channels is applied to the phase difference calculating part 312.

Further, as shown in FIG. 11B, in the case where the number Mⁿ of peak points extracted from two movement waveforms, respectively are different from each other (in FIG. 11B, there are four peak points () and three peak points (◯)), the inter-channel peak matching part 312c determines one peak point of one of movement waveforms (for example the peak point () of the channel one) as a reference peak point and determines peak points of another one of movement waveforms (for example the peak point (◯) of the channel two) as comparison peak points.

Next, the inter-channel peak matching part 312c calculates a time difference between the reference peak point () and each of comparison peak points (◯) and selects such one of the comparison peak points (◯) that the time difference is shortest for each reference point to match the peak points between the movement waveforms.

More specifically, the inter-channel peak matching part 312c sets “j” minimizing |P²ⱼ−P¹ᵢ| as m(i) in FIG. 11B to match P²ₘ₍ᵢ₎ to P¹ᵢ.

Next, information of the peak points matched between the channels is applied to the phase difference calculating part 312d.

In FIG. 11, was explained the case where peak points between two movement waveforms. However, matching peak points among more than two movement waveforms can be similarly made.

For example, in a case where the number Mⁿ extracted from more than two movement waveforms, respectively, are the same, the inter-channel peak matching part 312c sequentially matches peak points among the movement waveforms similarly to the case shown in FIG. 11A.

In addition, for example, in a case where the number of peak points Mⁿ extracted from more than two movement waveforms, respectively, are different, the inter-channel peak matching part 312c determines one peak point of one of more than two movement waveforms as a reference peak point and determines peak points of other movement waveforms as comparison peak points. Next, the inter-channel peak matching part 312c calculates time differences between the reference peak point and the comparison peak points and selects one of the comparison points which provides a minimum time difference from the reference peak point for each reference peak point to match the peak points among the movement waveforms.

### <Phase Difference Calculating Part>

The phase difference calculating part 312d calculates phase differences Θᵢ among a plurality of the movement waveforms on the basis of the peak time difference and the time differences of the peak points matched between channels. The phase difference Θᵢ calculated by the phase difference calculating part 312d according to the second embodiment corresponds to an instantaneous phase difference at the peak time (hereinafter, referred to as “instantaneous phase difference”).

For example, in the case where the phases are compared between two movement waveforms obtained with the movement sensor 6 including tapping devices of the channel one and two like the second embodiment, the instantaneous phase difference Θᵢ can be calculated by Equation (6).

Θᵢ=(P²ₘ₍ᵢ₎−P¹ᵢ)/R¹ᵢ×360 (6)

The term (P²ₘ₍ᵢ₎−P¹ᵢ) in Equation (6) represents the time difference of peak points matched between channels (inter-channel time difference).

Further, the instantaneous phase difference Θᵢ among more than two movement waveforms can be calculated by determining an instantaneous phase of one movement waveforms (for example, D¹(t)) as a reference and calculating differences from instantaneous phases of other movement waveforms (for example, D²(t), D³(t)).

### [Method of Comparing Phases]

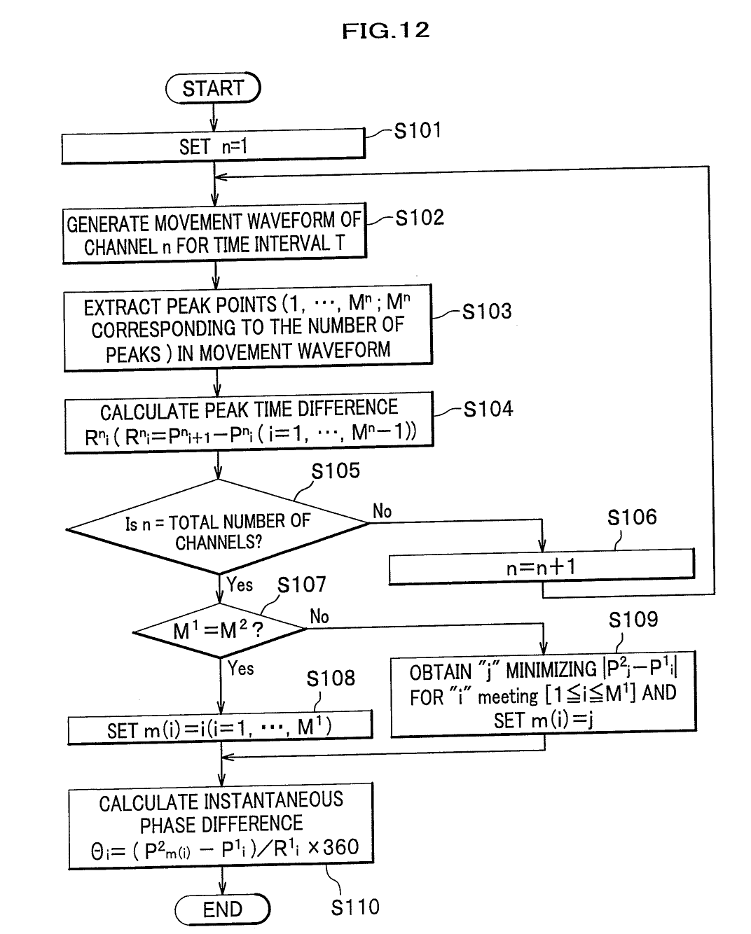

With reference to FIGS. 9 and 12, will be describe a method of comparing phases of a plurality of the movement waveforms with the moving body inspection apparatus 1 according to the second embodiment. FIG. 12 is a flowchart of a phase comparing process in the moving body inspection apparatus 1 according to the second embodiment.

First, the movement waveform generating part 211 of the analysis processing section 21 sets (n=1) before analyzing the waveform data obtained from the n channels of movement sensor 6 (see FIG. 1) (step S101).

Next, the movement waveform generating part 211 of the analysis processing section 21 generates the movement waveform having the time interval T on the basis of the waveform data of the “n” channel (step S102). Here, as described earlier, the time interval T is generally the measurement time interval by the movement sensor 6.

The phase comparing part 312 of the analysis processing section 21 extracts peak points (1, . . . , Mⁿ; Mⁿ corresponding to the number of peaks) in the movement waveform with the peak point extracting part 312a (step S103).

The phase comparing part 312 in the analysis processing section 21 calculates peak time difference Rⁿᵢ (Rⁿᵢ=Pⁿᵢ=Pⁿᵢ (i=1, . . . , Mⁿ−1)) from the time difference between peak points adjoining to each other in time in one movement waveform (step S104).

The movement waveform generating part 211 of the analysis processing section 21 determines whether (n=the total number of channels) (step S105). If (n≠the total number of channels)(No, in the step S105), the movement waveform generating part 211 returns to step S102 and repeats the process from the steps S102 to S106 until (n=the total number of channels). On the other hand, if (n=the total number of channels)(Yes, in the step S105), the movement waveform generating part 211 proceeds to step S107.

The phase comparing part 312 of the analysis processing section 21 determines whether the number of peak points are identical among a plurality of the movement waveforms with the inter-channel peak matching part 312c (step S107). If the number of peak points are identical (Yes, in step S107), the phase comparing part 312 sets m(i)=i (i=1, . . . , M¹) (step S108), and proceeds to a step S110. On the other hand, if the number of peaks are different (No, in step S107), the phase comparing part 312 obtains “j” minimizing |P²ⱼ−P¹ᵢ| and sets m(i)=j (step S109), and proceeds to step S110.

The phase comparing part 312 of the analysis processing section 21 can calculate the instantaneous phase differences among a plurality of the movement waveforms (step S110). Here, as described earlier, the instantaneous phase differences can be calculated with Equation (6).

### [Example of Display Screen Image]

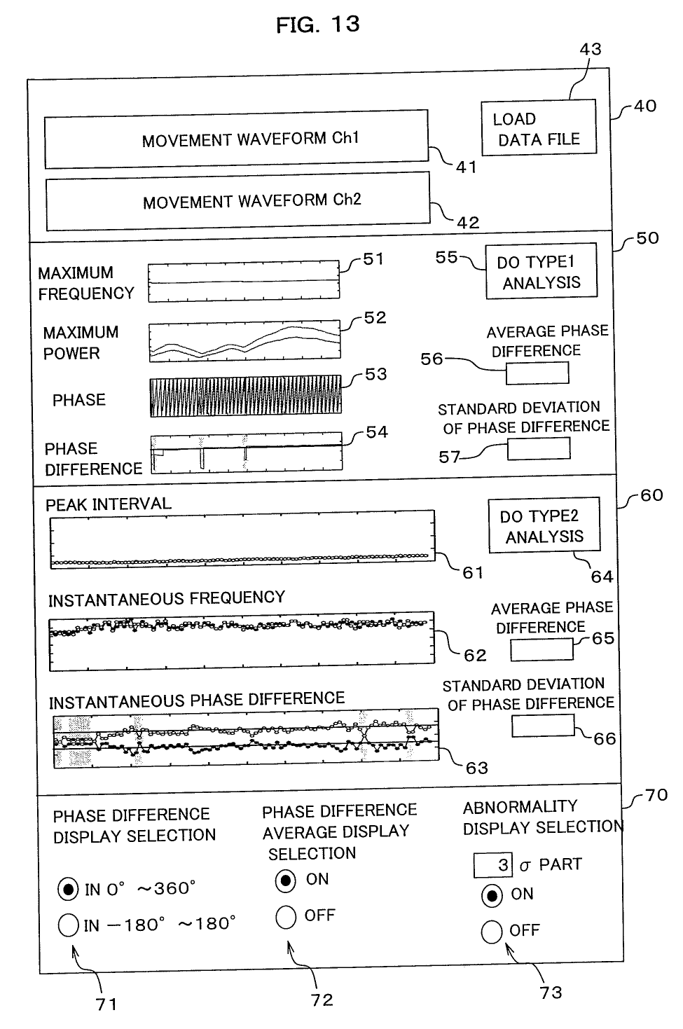

FIG. 13 shows an example of the screen image displayed on the display 4 with the display processing part 23 according to the first and second embodiments.

As shown in FIG. 13, the screen image displayed on the display 4 generally includes, for example, a movement waveform display area 40, a type-1 analysis display area 50 for displaying an analysis result according to the first embodiment, a type-2 analysis display area 60 for displaying an analysis result according to the second embodiment, and a phase difference display setting area 70 for setting a display format of the phase difference displayed in the type-1 analysis displayer area 50 and the type-2 analysis display area 60.

The movement waveform display area 40 displays, for example, the movement waveform 41 obtained in the channel one and the movement waveform 42 obtained by the channel two. This screen image can be displayed on the display 4 by depressing a load-data-file button 43 after measurement with the movement sensor 6. In addition, although not shown in FIG. 13, occasionally, a desired type of movement waveform can be additionally displayed after conversion.

The type-1 analysis display area 50 is provided for displaying the analysis result according to the first embodiment. For example, the maximum power frequency, the intensity at the maximum power frequency (represented with “MAXIMUM FREQUENCY” in FIG. 13), the phase at the maximum power frequency, the phase difference at the maximum power frequencies, of which methods of calculation were described in the first embodiment, are displayed in the display areas 51 to 54 as charts represented with time. This display screen image can be provided by depressing the do-type-1 analysis button 55 for the first analysis, so that the information processing section 2 including analysis processing section 21 and the display processing part 23 conducts the analysis process for the movement waveforms 41 and 42 and displays the analysis results on the display 4. Further, the information processor 2 can calculates an average and a standard deviation of the phase difference 54 of the displayed maximum power frequency to display the average and the standard deviation on the display areas 56 and 57, respectively.

The type-2 analysis display area 60 is provided for displaying the analysis result according to the second embodiment. For example, the peak time difference, the instantaneous frequency, the instantaneous phase difference, explained in description of the method of calculating in the second embodiment, on the display areas 61 to 63, respectively as sequential charts. This screen image can be made by depressing a do type-2-analysis button 64 for the second analysis by the operator. More specifically, the information processor 2 including the analysis processing section 21 and the display processing part 23 conducts analysis of the movement waveforms 41 and 42 to display the analysis result on the display 4. In addition the information processor 2 calculates an average and a standard deviation of the displayed instantaneous phase difference 63 to display the average and the standard deviation on display areas 65 and 66, respectively.

A phase difference display setting area 70 is provided for setting display formats of the phase difference display areas 54 and 63 displayed in the first analysis display area 50 and the second analysis display area 60. The phase difference display selection button 71 provides selection by the operator as to whether the longitudinal coordinate of the chart in the phase difference display areas 54 and 63 in a range from 0 degrees to 360 degrees or a range from −180 degrees to 180 degrees. This can display the phase difference waveform representing the phase difference at the maximum power frequency or the instantaneous phase difference at centers of the phase difference display areas 54 and 63 in both cases where the in-phase movement and anti-phase movement are analyzed.

The display selecting button 72 for displaying an average line of the phase difference is provided for selection by an operator as whether the average line in the phase difference is to be displayed on the phase difference display areas 54 and 64.

An abnormality display selection button 73 is provided for selection by the operator as to whether an abnormal part is to be displayed, which is caused by determining time zone meeting a predetermined condition (for example, a time zone exceeding a threshold) as an abnormal part. The abnormal part is displayed, for example, with a color different from those in other time zones. This gives the operator visual information which can indicate the part having difficulty in the movement of the subject.

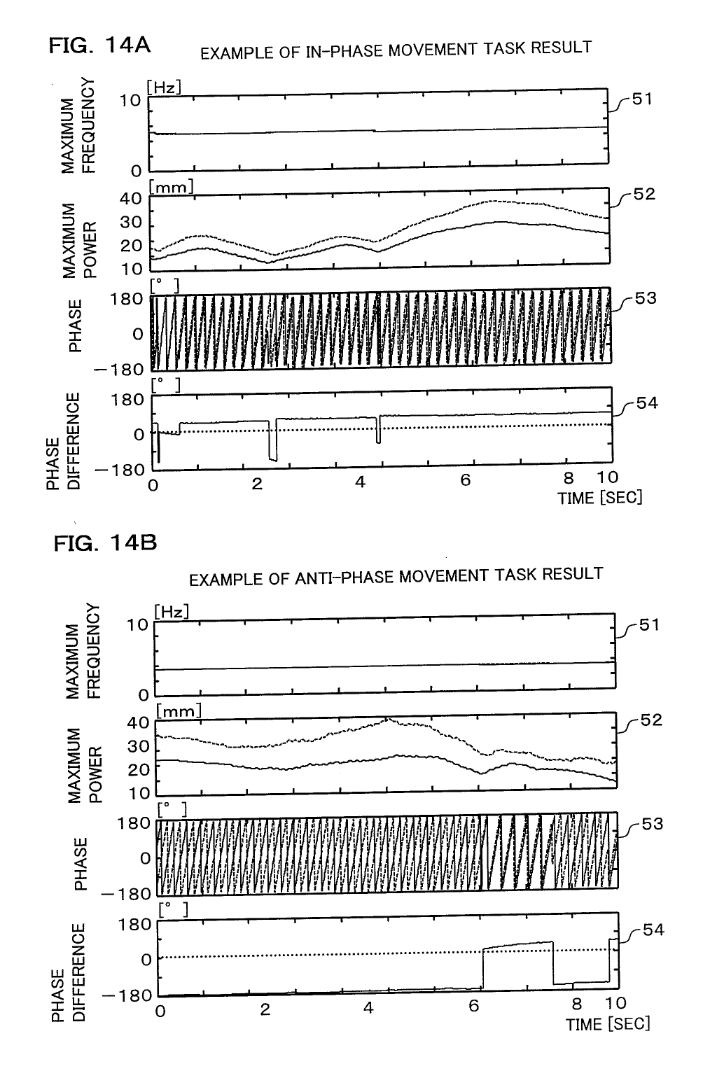

FIGS. 14A and 14B show examples of the analysis results according to the first embodiment, which is displayed in the type-1 analysis display area 50 on the display 4. The waveforms shown in FIGS. 14A and 14B are only examples and are not totally identical with the waveforms in FIG. 13.

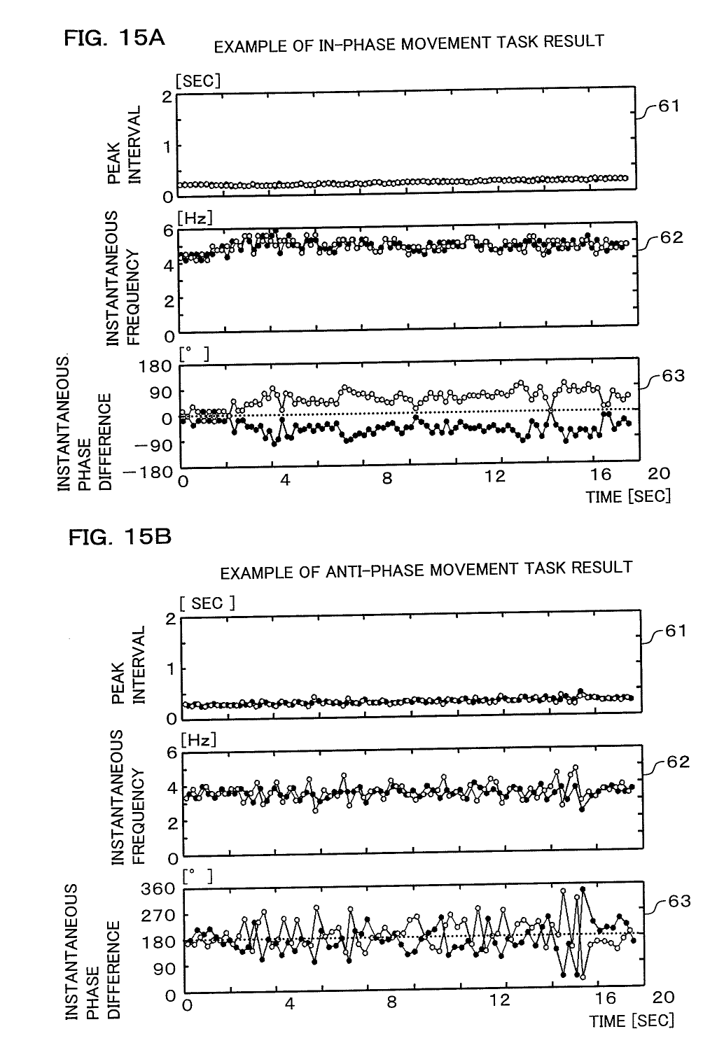

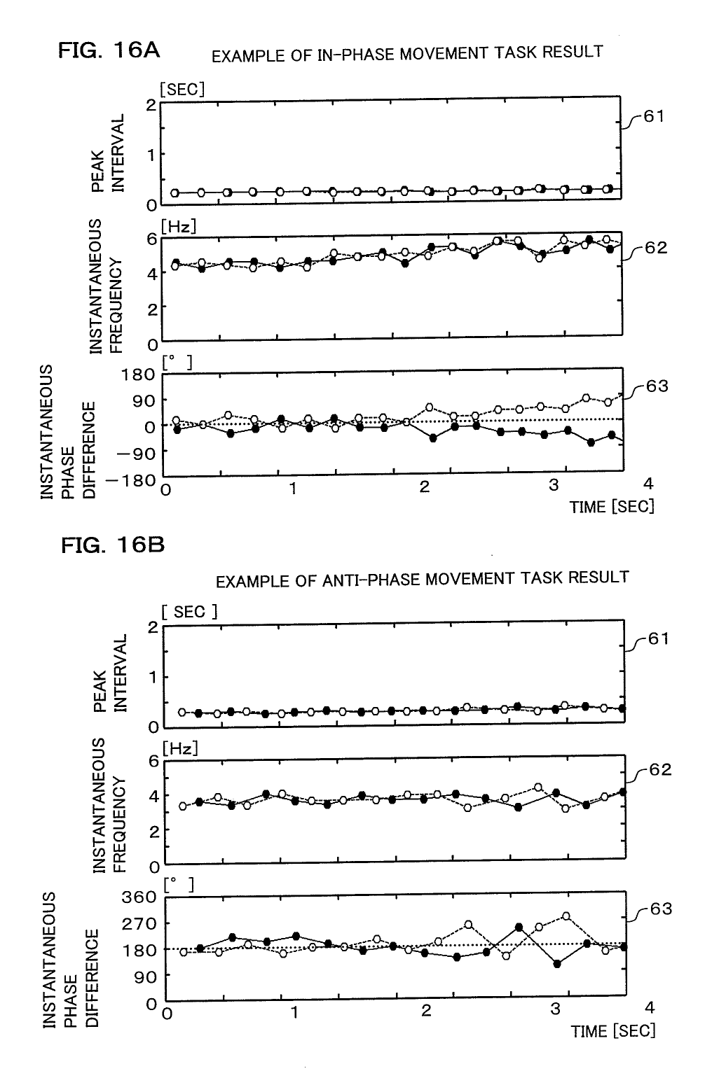

FIGS. 15A and 15B show examples of displayed analysis results according to the second embodiment, displayed on the type-2 analysis result display area 60 on the display 4. FIG. 15A shows a part of the second analysis result display area 60 in a case where a task of the in-phase movement is applied to the subject. In addition, the waveforms shown in FIGS. 15A and 15B are only examples and are not totally identical with the waveforms displayed on the type-2 analysis result display area 60 in FIG. 13. FIGS. 16A and 16B are enlarged views of parts shown in FIGS. 15A and 15B, respectively.

This process of displaying the analysis result on the display 4 can be conducted by the display processing section 23 with well-known programs for the analysis result of the movement waveforms. It is not necessary to display all analysis results on the same screen image. Thus, the display processing section 23 may display any of the analysis results selected by the operator.

Such displaying the analysis results on the display 4 gives an operator information of the motor function of the subject quantitatively and visually.

As described above, according to the first and second embodiments, the phases are compared among a plurality of movement waveforms. Thus, for example, in a case where the task of in-phase movement is applied to the subject, the operator can determine whether the motor function of the subject is normal by checking whether the phases are identical. Further, for example, in a case where the task of the in-phase movement is applied to the subject, the operator can determine whether the motor function of the subject is normal by checking whether the phase difference is always 180 degrees (whether the movements are alternately performed appropriately).

Thus, the moving body inspection apparatus 1 according to the present invention can provide preferable inspection of the motor function of the patients who have the trouble to the motor functions such as patients with cerebral infarction, Parkinson's disease patients, and cervical spine losis patients.

Further, according to the first embodiment, the phases can be compared among a plurality of movement waveforms without extracting peak points from the movement waveforms. More specifically, the analysis is not subject to affection of missing a peak points to be extracted, providing a stable analysis results. In addition, the analysis is performed for the frequency analysis interval having a predetermined time interval, providing the analysis results having low dispersion.

According to the second embodiment, the phase of each extracted peaks can be compared with the other one of a plurality of movement waveforms. In comparing phases, it is unnecessary to set the analysis interval having a predetermined duration, so that the entire time interval T of the measured movement waveform can be used for analysis.

Further, the operations according to the first and second embodiments can be selected or combined in accordance with the object of analysis, which provides a preferable comparison of phases among a plurality of movement waveforms.

This invention is not limited to the above-mentioned embodiments, but can be modified within the scope of the invention.

For example, in addition to outputting the analysis result as the analysis processing section 2 outputs, the analysis result output by the analysis processing section 2 may be subjected to a statistical process before outputting. In this case, a statistical processing section (not shown) is further provided in the information processor 2 which groups the analysis results on the basis of the subject information recorded in the subject data database (not shown)(for example, classifying the subjects into a normal group and a patient group), and conducts the statistical process to output, for example, calculation of averages, variances.

Further, in the first and second embodiments, the analysis such as the phase comparing is conducted after conversion of the output voltages measured by the movement sensor 6. However, the present invention is not limited to this, but the analysis may be conducted directly from the voltage output (waveform data).

In the first and second embodiments, the receiving coil 301 and the transmitting coil 302 in the movement sensor 6 are attached to the thumb and the index finger, but may be attached to any other fingers.

Further, the receiving coil 301 and the transmitting coil 302 may be attached to parts of the human body other than the fingers such as the eyelids, lips, arms, and feet.

In the first and second embodiments, the tapping device of a magnetic sensor type is used as the movement sensor 6, but any other movement sensor 6 is usable as long as the sensor can provide the waveform data indicative of the movement information. For example, the movement sensor 6 may be a well-known strain gage, accelerometer, or speed sensor and further may have a structure for providing the movement information by acquiring image data and analysis of the image data.

In addition, the method of comparing phases with the moving body inspection apparatus 1 can be provided by executing such a program with a general computer using an operating device and a storage in the computer. Thus, this invention is applicable to a program recording the method of comparing phases among a plurality of the movement waveforms.

1. A moving body inspection apparatus comprising:
an analyzer which analyzes sequential waveform data obtained from a movement sensor, the analyzer including:
a movement waveform generator which generates a plurality of waveforms from the waveform data, and
a phase comparator which compares phases among a plurality of movement waveforms; and
a display which displays a result of comparing phases;
wherein the movement waveform generator generates the movement waveforms in which each movement waveform includes at least one of a distance waveform, a speed waveform, an acceleration waveform, a jerk waveform, and a waveform convertible into the distance waveform, the speed waveform, the acceleration waveform, or the jerk waveform; and
wherein the phase comparator includes:
a peak detector which detects peaks in a plurality of the movement waveforms;
a peak interval calculator which calculates peak intervals between adjoining peaks which adjoin each other in time base out of the peaks,
an inter-waveform peak matcher which matches peaks among the plurality of the movement waveforms; and
a phase difference calculator which calculates a phase difference among the plurality of the movement waveforms.

2. The moving body inspection apparatus as claimed in claim 1, wherein when the number of peaks extracted from a plurality of the movement waveforms is the same, the inter-waveform peak matcher sequentially matches the peaks among a plurality of waveforms in time-order of the peaks.

3. The moving body inspection apparatus as claimed in claim 1, wherein when the number of the peaks extracted from a plurality of the movement waveforms is different, the inter-waveform peak matcher determines one of the peaks as a reference peak in one of the movement waveforms and matches the reference peak to one of the peaks in each of other waveforms which makes a time difference shortest.

4. A method of comparing phases among a plurality of movement waveforms obtained from a movement sensor, comprising: the steps of:
(a) extracting at least two peaks from each movement waveform of the plurality of movement waveforms;
(b) calculating peak time differences between the extracted adjacent peaks;
(c) matching the extracted peaks among the plurality of movement waveforms;
(d) calculating time difference between the peaks matched among the movement waveforms;
(e) calculating phase differences between the plurality of movement waveforms on the basis of the time differences between the matched peaks; and
(f) comparing phase differences between the movement waveforms.

5. The method as claimed in claim 4, wherein in the step (c), when the numbers of the peaks extracted from each movement waveform from the plurality of the waveforms are identical, the matching is a sequentially matching of the peaks extracted from each movement waveform from plurality of the movement waveforms.

6. The method as claimed in claim 4, wherein in the step (c), when the numbers of the peaks extracted from each movement waveform from the plurality of the waveforms are different, determining one of the peaks in selected one of the movement waveforms from the plurality of movement waveforms as a reference peak is performed, and wherein matching the peaks in the remaining movement waveforms of the plurality of movement waveforms to the reference peak is performed so as to make a time difference between the reference peak and the matched peaks in the remaining movement waveforms minimal.

7. The method as claimed in claim 4, wherein the method is implemented by a computer.
